(19) United States
(12) Patent Application Publication
(10) Pub. No.: US 2008/0227987 A1
(43) Pub. Date: Sep. 18, 2008

(54) Process for the Preparation of Onium Alkylsulfites

(76) Inventors: Nikolai (Mykola) Ignatyev, Duisburg (DE); Urs Welz-Biermann, Heppenheim (DE); Andriy Kucheryna, Kyiv (UA); Helge Willner, Muehlheim/Ruhr (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/995,444

(22) PCT Filed: Jun. 14, 2006
(86) PCT No.: PCT/EP06/05742

§ 371 Date: Jan. 11, 2008

(30) Foreign Application Priority Data
Jul. 14, 2005 (DE) 10 2005 032 837.7

(51) Int. Cl. C07D 233/56; C07C 209/00; C07C 211/63

(52) U.S. Cl. 548/335.1; 564/296; 564/291

(57) ABSTRACT

The invention relates to a process for the preparation of onium alkylsulfites by reaction of an onium halide or carboxylate with a symmetrically substituted dialkyl sulfite or with an asymmetrically substituted dialkyl sulfite at temperatures of 0 to 70° C.

The invention relates to a process for the preparation of onium alkylsulfites by reaction of an onium halide or carboxylate with a symmetrically substituted dialkyl sulfite or with an asymmetrically substituted dialkyl sulfite at temperatures of 0 to 70° C., and to alkylsulfites prepared by this process.

A large number of onium salts, for example alkylsulfates, are ionic liquids. Due to their properties, ionic liquids represent an effective alternative to traditional volatile organic solvents for organic synthesis in morn research. The use of ionic liquids as novel reaction medium could furthermore be a practical solution both to solvents emission and also to problems in the reprocessing of catalysts (R. Sheldon, Chem. Commun., 2001, pp. 2399-2407).

Ionic liquids or liquid salts are ionic species which consists of an organic cation and a generally inorganic anion. They do not contain any neutral molecules and usually have melting points below 373 K. However, the melting point may also be higher without limiting the utility of the salts in all areas of application. Examples of organic cations are, inter alia, tetraalkylammonium, tetraalkylphosphonium, N-alkylpyridinium, 1,3-dialkylimidazolium or trialkylsulfonium. Amongst a multiplicity of suitable anions, mention may be made, for example, of BF₄⁻, PF₆⁻, SbF₆⁻, NO₃⁻, CF₃SO₃⁻, (CF₃SO₂)₂N⁻, alkylSO₃⁻, arylSO₃⁻, CF₃CO₂⁻, CH₃CO₂⁻ or Al₂Cl₇⁻.

Ionic liquids with alkylsulfite or arylsulfite anions are unknown. Only occasional onium salts with a methylsulfite anion are described in the literature, for example trimethylphenylammonium methylsulfite (W. Voss and E. Blanke, Justus Liebigs Ann. Chem., 485 (1931), pp. 258-279) or tetrabutylammonium methylsulfite (M. Julia et al., Tetrahedron, 42 (1986), pp. 3841-3850). In addition, there is no general method for the preparation of onium alkylsulfites.

The object of the present invention was accordingly to provide a process for the preparation of onium alkylsulfites which results in alkylsulfites having a freely selectable substitution pattern in the cation and anion, and the provision of onium alkylsulfites.

The object is achieved by the process according to the invention. The invention therefore relates to a process for the preparation of onium alkylsulfites by reaction of an onium halide or onium carboxylate with a symmetrically substituted dialkyl sulfite or with an asymmetrically substituted dialkyl sulfite at temperatures of 0 to 70° C. The process according to the invention has the advantage that an alkyl group is not transferred from the sulfite employed to the cation, and the substitution pattern in the cation is thus independent of the choice of alkanesulfite anion. In addition, the dialkyl sulfites employed are less toxic than alkylsulfate esters. The byproducts obtained in the process according to the invention are alkyl halides or esters, which can themselves be employed as valuable reagents. The alkyl halides forming as by-product on use of onium halides are, in addition, generally gases or readily volatile compounds, which can be removed from the reaction mixture without major process-engineering effort.

It is of course possible to use both symmetrically substituted dialkyl sulfites having 1 to 10 C atoms or to use asymmetrically substituted dialkyl sulfites having 1 to 10 C atoms or even more highly alkylated starting materials in the process according to the invention.

Onium halides which are suitable for the process according to the invention are ammonium halides, phosphonium halides, thiouronium halides, guanidinium halides or halides with a heterocyclic cation, where the halides can be selected from the group fluorides, chlorides or bromides. Ammonium, phosphonium, guanidinium halides or halides with a heterocyclic cation are preferably employed in the process according to the invention.

Onium carboxylates which are suitable for the process according to the invention are ammonium carboxylates, phosphonium carboxylates, thiouronium carboxylates, guanidinium carboxylates or carboxylates with a heterocyclic cation, where the carboxylates can be selected from the group of the carbonates, formates, acetates, propylates or butylates. Ammonium, phosphonium, guanidinium carboxylates or carboxylates with a heterocyclic cation are preferably employed in the process according to the invention, in particular ammonium, phosphonium, guanidinium acetates or acetates with a heterocyclic cation.

Onium halides are preferably employed in the processes according to the invention.

The onium halides or carboxylates are generally commercially available or can be prepared by synthetic methods as are known from the literature, for example in the standard works, such as Houben-Weyl, Methoden der organischen Chemie [Methods of Organic Chemistry], Georg-Thieme-Verlag, Stuttgart, or Richard C. Larock, Comprehensive Organic Transformations, 2nd Edition, Wiley-VCH, New York, 1999. Use may also be made here of variants known per se which are not mentioned here in greater detail.

Ammonium or phosphonium halides can be described, for example, by the formula (1)

[XR₄]⁺Hal⁻ (1),

where
X denotes N, P
Hal denotes F, Cl or Br and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms,
which may be substituted by alkyl groups having 1-6 C atoms,
where one or more R may be partially substituted by Cl, Br and/or CN or partially or fully by F, or F and Cl, or F and Br, or F, Cl and Br, but where all four or three R cannot be fully substituted by halogens,
and where one or two non-adjacent carbon atoms of the R which are not in the α- or ω-position may be replaced by atoms and/or atom groups selected from the group —O—, —C(O)—, —S—, —S(O)— or —SO₂—.

Accordingly, compounds of the formula (1) in which all four or three substituents R are fully substituted by halogens, for example tris(trifluoromethyl)methylammonium chloride, tetra(trifluoromethyl)ammonium chloride or tetra(nonafluorobutyl)phosphonium chloride, are excluded.

Guanidinium halides can be described, for example, by the formula (2)

[C(NR¹R²)(NR³R⁴)(NR⁵R⁶)]⁺Hal⁻ (2),

where
Hal denotes F, Cl or Br and
R¹ to R⁶ each, independently of one another, denote hydrogen or CN,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms,
which may be substituted by alkyl groups having 1-6 C atoms,
where one or more of the substituents R¹ to R⁶ may be partially substituted by NO₂, CN, Cl and/or Br, partially or fully by F, or F and Cl, or F and Br or F, Cl and Br, but where all substituents on an N atom cannot be fully substituted by halogens,
where the substituents R¹ to R⁶ may be connected to one another in pairs by a single or double bond and
where, in the substituents R¹ to R⁶, one or two non-adjacent carbon atoms which are not bonded directly to the heteroatom and are not in the ω-position may be replaced by atoms and/or atom groups selected from the group —O—, —C(O)—, —S—, —S(O)— or —SO₂—.

Thiouronium halides can be described, for example, by the formula (3)

[(R¹R²N)—C(═SR⁷)(NR³R⁴)]⁺Hal⁻ (3),

where
Hal denotes Cl or Br, and
R¹ to R⁴ and R⁷ each, independently of one another, denote hydrogen or CN, where hydrogen is excluded for R⁷,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more of the substituents R¹ to R⁴ and R⁷ may be partially or fully substituted by F, Cl and/or Br, in particular —F and/or —Cl, or partially by CN, but where all substituents on an N atom cannot be fully substituted by halogens,
where the substituents R¹ to R⁴ and R⁷ may be connected to one another in pairs by a single or double bond and where, in the substituents R¹ to R⁴ and R⁷, one or two non-adjacent carbon atoms which are not bonded directly to the heteroatom and are not in the w-position may be replaced by atoms and/or atom groups selected from the group —O—, —C(O)—, —S—, —S(O)— or —SO₂—.

Halides with a heterocyclic cation can be described, for example, by the formula (4)

[HetN]⁺Hal⁻ (4),

where
Hal denotes Cl or Br and
HetN⁺ denotes a heterocyclic cation selected from the group

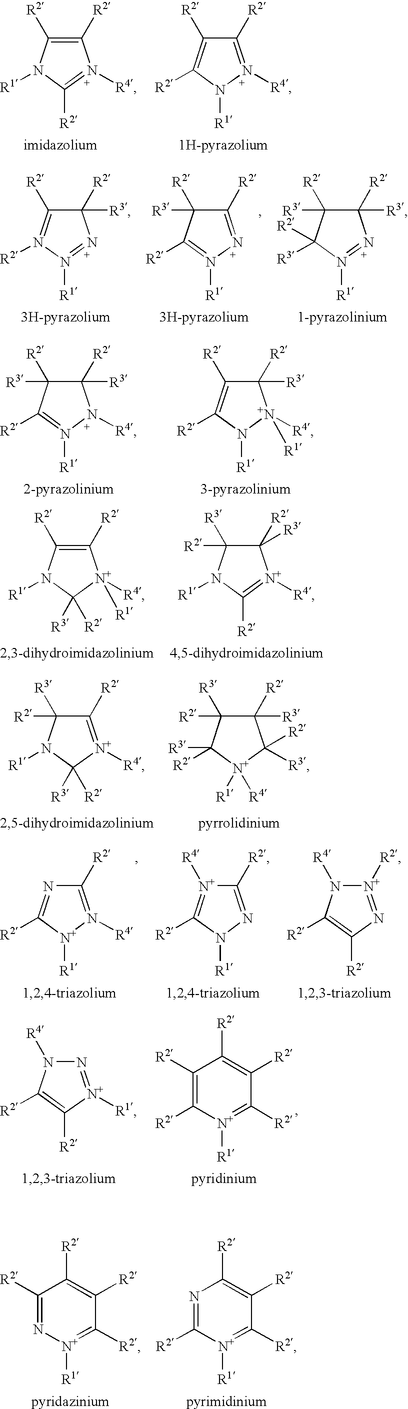

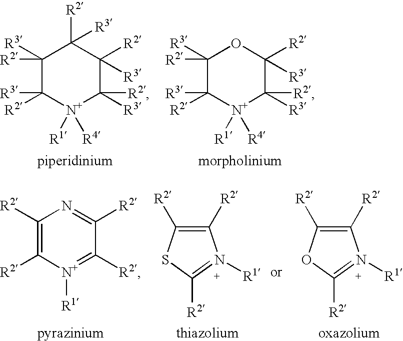

where the substituents
R¹′ to R⁴′ each, independently of one another, denote hydrogen or CN,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
dialkylamino containing alkyl groups having 1-4 C atoms, which, however, is not bonded to the heteroatom of the heterocycle,
saturated, partially or fully unsaturated cycloalkyl having 3-7 C atoms, which may be substituted by alkyl groups having 1-6 C atoms, or aryl-C₁-C₆-alkyl,
where the substituents R¹′ and R⁴′ may be partially or fully substituted by F, Cl and/or Br, in particular —F and/or —Cl, but where R¹′ and R⁴′ are not simultaneously CN or cannot simultaneously be fully substituted by F or other halogens,
where the substituents R²′ and R³′ may be partially or fully substituted by F, Cl and/or Br, in particular —F and/or —Cl, or partially by NO₂ or CN
and where, in the substituents R¹′ to R⁴′, one or two non-adjacent carbon atoms which are not bonded directly to the heteroatom and are not in the ω-position may be replaced by atoms and/or atom groups selected from the group —O—, —C(O)—, —S—, —S(O)— or —SO₂—.

The C₁-C₂₀-alkyl group is, for example, methyl, ethyl, isopropyl, propyl, butyl, sec-butyl or tert-butyl, furthermore also pentyl, 1-, 2- or 3-methylbutyl, 1,1-, 1,2- or 2,2-dimethylpropyl, 1-ethylpropyl, hexyl, heptyl, octyl, nonyl, decyl, undecyl, dodecyl, tridecyl or tetradecyl, optionally fluorinated alkyl groups, for example difluoromethyl, trifluoromethyl, tetrafluoroethyl, pentafluoroethyl, heptafluoropropyl or nonafluorobutyl.

A straight-chain or branched alkenyl having 2 to 20 C atoms, in which a plurality of double bonds may also be present, is, for example, vinyl, allyl, 2- or 3-butenyl, isobutenyl, sec-butenyl, furthermore 4-pentenyl, isopentenyl, hexenyl, heptenyl, octenyl, —C₉H₁₇, —C₁₀H₁₉ to —C₂₀H₃₉; preferably vinyl, allyl, 2- or 3-butenyl, isobutenyl, sec-butenyl, furthermore preferably 4-pentenyl, isopentenyl or hexenyl.

A straight-chain or branched alkynyl having 2 to 20 C atoms, in which a plurality of triple bonds may also be present, is, for example, ethynyl, 1- or 2-propynyl, 2- or 3-butynyl, furthermore 4-pentynyl, 3-pentynyl, hexynyl, heptynyl, octynyl, —C₉H₁₅, —C₁₀H₁₇ to —C₂₀H₃₇, preferably ethynyl, 1- or 2-propynyl, 2- or 3-butynyl, 4-pentynyl, 3-pentynyl or hexynyl.

For the purposes of the present invention, fully unsaturated cycloalkyl is also taken to mean aromatic substituents.

Unsubstituted saturated or partially or fully unsaturated cycloalkyl groups having 3-7 C atoms are therefore cyclopropyl, cyclobutyl, cyclopentyl, cyclohexyl, cycloheptyl, cyclopentenyl, cyclopenta-1,3-dienyl, cyclohexenyl, cyclohexa-1,3-dienyl, cyclohexa-1,4-dienyl, phenyl, cycloheptenyl, cyclohepta-1,3-dienyl, cyclohepta-1,4-dienyl or cyclohepta-1,5-dienyl, each of which may be substituted by C₁- to C₆-alkyl groups, where the cycloalkyl group or the cycloalkyl group which is substituted by C₁- to C₆-alkyl groups may in turn also be substituted by F or F and Cl. However, the cycloalkyl groups may likewise be substituted by further functional groups, for example by CN, SO₂R′, SO₂OR′, SO₂NH₂, C(O)NH₂ or C(O)OR′. R′ here has a meaning defined below.

Aryl-C₁-C₆-alkyl denotes, for example, benzyl, phenylethyl, phenylpropyl, phenylbutyl, phenylpentyl or phenylhexyl, where both the phenyl ring and also the alkylene chain may be partially or fully substituted as described above by F or F and Cl, particularly preferably benzyl or phenylpropyl. However, the phenyl ring or also the alkylene chain may likewise be substituted by further functional groups, for example by CN, SO₂R′, SO₂OR′, SO₂NH₂, C(O)NH₂ or C(O)OR′.

R′ denotes non-, partially or perfluorinated C₁- to C₆-alkyl, C₃- to C₇-cycloalkyl, unsubstituted or substituted phenyl. In R′, C₃- to C₇-cycloalkyl is, for example, cyclopropyl, cyclobutyl, cyclopentyl, cyclohexyl or cycloheptyl.

In R′, substituted phenyl denotes phenyl which is substituted by C₁- to C₆-alkyl, C₁- to C₆-alkenyl, NO₂, F, Cl, Br, I, C₁-C₆-alkoxy, SCF₃, SO₂CF₃, SO₂CH₃, COOR″, SO₂X′, SO₂NR″₂ or SO₃R″, where X′ denotes F, Cl or Br and R″ denotes a non- or partially fluorinated C₁- to C₆-alkyl or C₃- to C₇-cycloalkyl as defined for R′, for example o-, m- or p-methylphenyl, o-, m- or p-ethylphenyl, o-, m- or p-propylphenyl, o-, m- or p-isopropylphenyl, o-, m- or p-nitrophenyl, o-, m- or p-methoxyphenyl, o-, m- or p-ethoxyphenyl, o-, m-, p-(trifluoromethyl)phenyl, o-, m-, p-(trifluoromethoxy)phenyl, o-, m-, p-(trifluoromethylsulfonyl)phenyl, o-, m- or p-fluorophenyl, o-, m- or p-chlorophenyl, o-, m- or p-bromophenyl, o-, m- or p-iodophenyl, furthermore preferably 2,3-, 2,4-, 2,5-, 2,6-, 3,4- or 3,5-dimethylphenyl, 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3-, 2,4-, 2,5-, 2,6-, 3,4- or 3,5-dimethoxyphenyl, 5-fluoro-2-methylphenyl, 3,4,5-trimethoxyphenyl or 2,4,5-trimethylphenyl.

In the substituents R, R¹ to R⁷ or R¹′ to R⁴′, one or two non-adjacent carbon atoms which are not bonded directly to the heteroatom and are not in the w-position may also be replaced by atoms and/or atom groups selected from the group —O—, —C(O)—, —S—, —S(O)— or —SO₂—.

Without restricting generality, examples of substituents R, R¹ to R⁷ and R¹′ to R⁴′ modified in this way are:

—OCH₃, —OCH(CH₃)₂, —CH₂OCH₃, —CH₂—CH₂—O—CH₃, —C₂H₄OCH(CH₃)₂, —C₂H₄SC₂H₅, —C₂H₄SCH(CH₃)₂, —S(O)CH₃, —SO₂CH₃, —SO₂C₆H₅, —SO₂C₃H₇, —SO₂CH(CH₃)₂, —SO₂CH₂CF₃, —CH₂SO₂CH₃, —O—C₄H₈—O—C₄H₉, —CF₃, —C₂F₅, —C₃F₇, —C₄F₉, —CF₂CF₂H, —CF₂CHFCF₃, —CF₂CH(CF₃)₂, —C₂F₄N(C₂F₅)C₂F₅, —CHF₂, —CH₂CF₃, —C₂F₂H₃, —C₃FH₆, —CH₂C₃F₇, —CH₂C(O)OCH₃, —CH₂C(O)CH₃, —CH₂C₆H₅ or —C(O)C₆H₅.

The substituents R¹ to R⁷ are each, independently of one another, preferably a straight-chain or branched alkyl group having 1 to 10 C atoms. The substituents R¹ and R², R³ and R⁴ and R⁵ and R⁶ in compounds of the formulae (2) and (3) may be identical or different here.

R¹ to R⁷ are particularly preferably each, independently of one another, methyl, ethyl, n-propyl, isopropyl, n-butyl, sec-butyl, phenyl or cyclohexyl, very particularly preferably methyl, ethyl, n-propyl, isopropyl or n-butyl. However, the substituents R and R¹ to R⁷ may likewise be substituted by further functional groups, for example by CN, SO₂R′, SO₂OR′, SO₂NH₂, SO₂NR′₂, C(O)NH₂, C(O)NR′₂ or C(O)OR′. R′ here has a meaning defined above.

In accordance with the invention, suitable substituents R¹′ to R⁴′ of compounds of the formula (4), besides hydrogen, are preferably: CN, C₁- to C₂₀-, in particular C₁- to C₁₂-alkyl groups, and saturated or unsaturated, i.e. also aromatic, C₃- to C₇-cycloalkyl groups, each of which may be substituted by C₁- to C₆-alkyl groups, in particular phenyl or aryl-C₁-C₆-alkyl.

The substituents R¹′ and R⁴′ are each, independently of one another, particularly preferably CN, methyl, ethyl, isopropyl, propyl, butyl, sec-butyl, pentyl, hexyl, octyl, decyl, cyclohexyl, phenyl, phenylpropyl or benzyl. They are very particularly preferably methyl, CN, ethyl, n-butyl or hexyl. In pyrrolidinium or piperidinium compounds, the two substituents R¹′ and R⁴′ are preferably different.

The substituent R²′ or R³′ is in each case, independently of one another, in particular hydrogen, dimethylamino, diethylamino, methyl, ethyl, isopropyl, propyl, butyl, sec-butyl, cyclohexyl, phenyl or benzyl. R² is particularly preferably hydrogen, methyl, ethyl, isopropyl, propyl, butyl, sec-butyl or dimethylamino. R²′ and R³′ are very particularly preferably hydrogen.

In an embodiment, the alkyl groups as substituents R and R¹ to R⁶ and R¹′ and R⁴′ of the heterocyclic cations of the formula (4) are different from the alkyl group of the anion in the onium alkylsulfite.

However, the onium alkylsulfite prepared in accordance with the invention may also have alkyl groups in the cation which are identical with the alkyl group in the anion, but have not been introduced in accordance with the invention by alkylation.

Up to four substituents of the guanidinium cation [C(NR¹R²)(NR³R⁴)—(NR⁵R⁶)]⁺ may also be connected in pairs in such a way that mono-, bi- or polycyclic cations form.

Without restricting generality, examples of guanidinium cations of this type are:

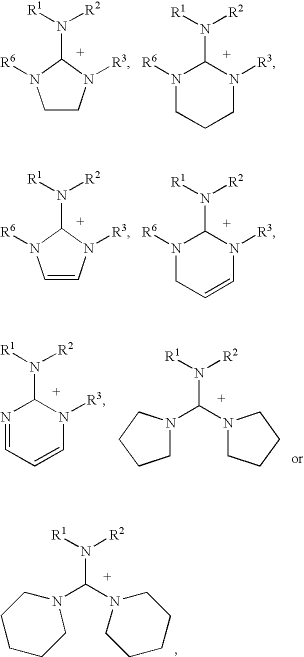

where the substituents R¹ to R³ and R⁶ can have an above-mentioned or particularly preferred meaning.

The carbocycles or heterocycles of the guanidinium cations indicated above may optionally also be substituted by C₁- to C₆-alkyl, C₁- to C₆-alkenyl, NO₂, F, Cl, Br, I, C₁-C₆-alkoxy, SCF₃, SO₂CF₃, SO₂CH₃, CN, COOR″, SO₂NR″₂, SO₂X′ or SO₃R″, where X′ denotes F, Cl or Br and R″ denotes a non- or partially fluorinated C₁- to C₆-alkyl or C₃- to C₇-cycloalkyl as defined for R′, or by substituted or unsubstituted phenyl.

Up to four substituents of the thiouronium cation [(R¹R²N)—C(═SR⁷)(NR³R⁴)]⁺ may also be connected in pairs in such a way that mono-, bi- or polycyclic cations form.

Without restricting generality, examples of cations of this type are indicated below:

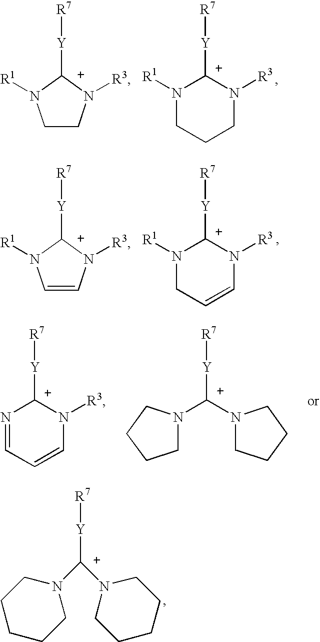

where Y═S and the substituents R¹, R³ and R⁷ can have an above-mentioned or particularly preferred meaning.

The carbocycles or heterocycles of the thiouronium cations indicated above may optionally also be substituted by C₁- to C₆-alkyl, C₁- to C₆-alkenyl, NO₂, F, Cl, Br, I, C₁-C₆-alkoxy, SCF₃, SO₂CF₃, SO₂CH₃, COOR″, SO₂NR″₂, SO₂X′ or SO₃R″, where X′ denotes F, Cl or Br and R″ denotes a non-, partially or perfluorinated C₁- to C₆-alkyl or C₃- to C₇-cycloalkyl as defined for R′, or by substituted or unsubstituted phenyl.

However, the substituents R¹′ to R⁴′ may likewise be substituted by further functional groups, for example by CN, SO₂R′, SO₂OR′ or COOR′. R′ here has a meaning defined above.

HetN⁺ of the formula (4) is preferably

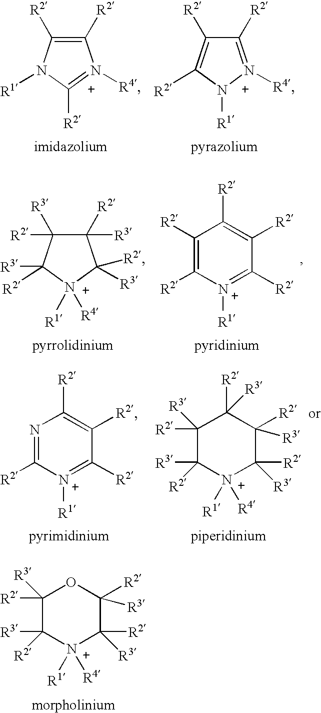

where the substituents R¹′ to R⁴′ each, independently of one another, have a meaning described above.

HetN⁺ is particularly preferably imidazolium, pyrrolidinium or pyridinium, as defined above, where the substituents R¹′ to R⁴′ each, independently of one another, have a meaning described above.

In the said formulae (1) to (4), Hal may in accordance with the invention be replaced by carboxylates, as defined above. The choice of the anion does not restrict the choice of the cation.

The symmetrically substituted dialkyl sulfite employed in the processes according to the invention is preferably a dialkyl sulfite containing a straight-chain or branched alkyl group having 1-10 C atoms, preferably having 1-4 C atoms, particularly preferably having 1-2 C atoms. The alkyl group is preferably a straight-chain or branched alkyl group having 1-4 C atoms, such as, for example, methyl, ethyl, isopropyl, propyl, butyl, sec-butyl or tert-butyl. The alkyl group is preferably methyl or ethyl.

Examples of symmetrically substituted dialkyl sulfites are dimethyl sulfite, diethyl sulfite, di-(n-propyl) sulfite, di-(isopropyl) sulfite, di-(n-butyl) sulfite or di-(sec-butyl) sulfite. Preference is given to the use of dimethyl sulfite or diethyl sulfite.

The symmetrical dialkyl sulfites employed are generally commercially available or can be prepared by synthetic methods as are known from the literature, for example in the standard works, such as Houben-Weyl, Methoden der organischen Chemie [Methods of Organic Chemistry], Georg-Thieme-Verlag, Stuttgart, or Richard C. Larock, Comprehensive Organic Transformations, 2nd Edition, Wiley-VCH, New York, 1999, or from the article by W. Voss and E. Blanke, Justus Liebigs Ann. Chem., 485 (1931), pp. 258-279. Use can also be made here of variants known per se which are not mentioned here in greater detail.

The asymmetrically substituted dialkyl sulfite employed is preferably a dialkyl sulfite containing a straight-chain or branched alkyl group having 1 to 10 C atoms and a methyl or ethyl group as second alkyl group, preferably containing an alkyl group having 3-8 C atoms. Examples of asymmetrically substituted dialkyl sulfites are methyl propyl sulfite, methyl butyl sulfite, ethyl butyl sulfite, methyl pentyl sulfite, ethyl pentyl sulfite, methyl hexyl sulfite, ethyl hexyl sulfite, methyl heptyl sulfite, ethyl heptyl sulfite, methyl octyl sulfite or ethyl octyl sulfite.

The asymmetrical dialkyl sulfites employed can be prepared by synthetic methods as are known from the literature, for example in the standard works, such as Houben-Weyl, Methoden der organischen Chemie [Methods of Organic Chemistry], Georg-Thieme-Verlag, Stuttgart, or Richard C. Larock, Comprehensive Organic Transformations, 2nd Edition, Wiley-VCH, New York, 1999, or from the article by W. Voss and E. Blanke, Justus Liebigs Ann. Chem., 485 (1931), pp. 258-279, or by V. M. Pavlov, J. Gen. Chem. USSR (Eng. transl.), 41 (1971), pp. 2559-2561. Use can also be made here of variants known per se which are not mentioned here in greater detail.

A general scheme summarises the process according to the invention, where the arrow in the case of the alkyl-halogen compound forming represents a symbol for the volatility of the compound:

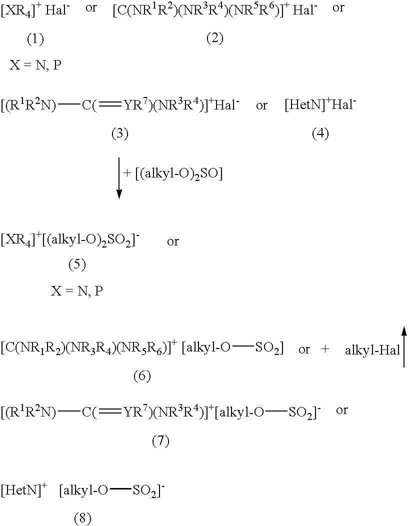

The substituents R, R¹ to R⁷ and HetN⁺ of the compounds of the formulae (1) to (8) correspond to the meanings as described above.

The reaction with dialkyl sulfites is carried out at temperatures between 0 and 70° C., preferably at temperatures between 20 and 60° C. and particularly preferably at room temperature. The choice of the optimum reaction temperature depends here on the level of the excess of the dialkyl sulfite and on the type of halide and dialkyl sulfite employed. In general, higher temperatures and longer reaction times are necessary in the case of dialkyl sulfites containing relatively long alkyl chains.

No solvent is required. However, it is also possible to employ solvents, for example dimethoxyethane, acetonitrile, acetone, tetrahydrofuran, dimethylformamide, dimethyl sulfoxide, dioxane, propionitrile, dichloromethane or mixtures with one another.

The reaction is carried out with an excess or equimolar amount of dialkyl sulfite.

Alternatively, onium salts with alkylsulfite anions can be prepared by direct alkylation of organic bases using dialkyl sulfites. The experiments on the alkylation of dimethylaniline and pyridine using dimethyl sulfite with formation of the corresponding methylsulfonates are described in the article by W. Voss and E. Blanke, Justus Liebigs Ann. Chem., 485 (1931), pp. 258-279. The corresponding formation of the methylsulfites in low yield is assumed, but not proven. The disadvantage of this method is the formation of onium compounds which have the same alkyl group both in the cation and in the anion and thus restrict the possible variety of the substitution patterns.

The present invention likewise relates to onium alkylsulfites prepared by the process according to the invention. Some of the compounds are known. Use of the process according to the invention simplifies access to these compounds and thus increases the possibility of utilisation of these compounds.

In addition, the present invention likewise relates to onium alkylsulfites of the formula (9), obtainable by the process according to the invention

[Kt]⁺[alkyl-O—SO₂]⁻ (9)

where
Kt denotes asymmetrical tetraalkylammonium, symmetrical and asymmetrical phosphonium, guanidinium, thiouronium cations or heterocyclic cations, where N-methylpyridinium sulfite is excluded. Corresponding cations have already been mentioned in the description of the suitable onium halides.

It goes without saying to the person skilled in the art that all substituents mentioned above, such as, for example, H, N, O, Cl, F, may be replaced by the corresponding isotopes.

The present invention likewise relates to the use of the compounds of the formula (9) according to the invention as solvent, solvent additive, heat-transfer medium, reducing agent, antioxidant, phase-transfer catalyst, as extractant, as additive, as surface-active substance, as electrolyte in electrochemical cells, as modifier or as plasticiser.

In addition, the compounds of the formula (9) are also suitable as starting material for the synthesis of onium salts with alkylsulfate anions by oxidation. This process represents an advantageous alternative for the preparation of onium salts with alkylsulfate anions, which has the advantage over conventional direct alkylation of organic bases using dialkyl sulfates, for example dimethyl sulfate, that the use of highly toxic dialkyl sulfate is unnecessary. Suitable oxidants in this reaction are all oxidants known to the person skilled in the art, for example oxygen.

In the case of the use as solvent or solvent additive, the compounds according to the invention are suitable in any type of reaction known to the person skilled in the art, for example reactions catalysed by transition metals, enzymes or other biocatalysts, such as, for example, hydroformylation reactions, oligomerisation reactions, C—C bond formation reactions, for example the Heck coupling, but also esterifications, isomerisation reactions or reactions for the formation of amide bonds.

Even without further comments, it is assumed that a person skilled in the art will be able to utilise the above description in the broadest scope. The preferred embodiments and examples should therefore merely be regarded as descriptive disclosure which is absolutely not limiting in any way.

The NMR spectra were measured on solutions in deuterated solvents at 20° C. on a Bruker ARX 400 spectrometer with a 5 mm ¹H/BB broad-band head with deuterium lock, unless indicated otherwise in the examples. The measurement frequencies of the various nuclei are: ¹H, 400.13 MHz and ³¹P: 161.98 MHz. The referencing method is indicated separately for each spectrum or each data set.

### EXAMPLES

### Example 1

### 1-Butyl-3-methylimidazolium methylsulfite

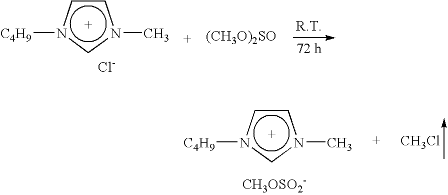

A mixture of 8.81 g (50.4 mmol) of 1-butyl-3-methylimidazolium chloride and 5.56 g (50.5 mmol) of dimethyl sulfite is stirred at room temperature for 72 hours under an inert-gas atmosphere (nitrogen) in a sealed 100 ml reaction vessel. The end of the reaction is determined by NMR measurement. The product is pumped off over the course of 1 hour in vacuo at 13.3 Pa and 60° C. (temperature of the oil bath), giving 11.8 g of liquid 1-butyl-3-methylimidazolium methylsulfite. The yield is virtually quantitative. The product is investigated by means of NMR spectroscopy.

¹H NMR (reference: TMS; solvent: CD₃CN), ppm: 0.88 t (CH₃); 1.28 m (CH₂); 1.79 m (CH₂); 3.17 s (CH₃O); 3.86 s (CH₃); 4.19 t (CH₂); 7.54 d,d (CH); 7.56 d,d (CH); 9.66 br. s. (CH); ³J_{H,H}=7.1 Hz; J_{H,H}=1.5 Hz.

### Example 2

### 1-Butyl-3-methylimidazolium ethylsulfite

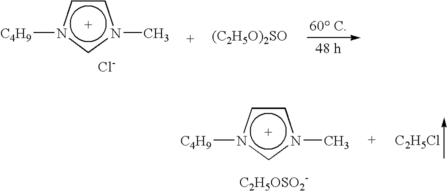

A mixture of 6.38 g (36.5 mmol) of 1-butyl-3-methylimidazolium chloride and 6.66 g (48.2 mmol) of diethyl sulfite is stirred at 60-70° C. (temperature of the oil bath) for 48 hours under an inert-gas atmosphere (nitrogen) in a sealed reaction vessel with pressure valves for 1-1.5 bar above atmospheric pressure. The end of the reaction is determined by NMR measurement. The product is pumped off over the course of 2 hours in vacuo at 13.3 Pa and 60° C. (temperature of the oil bath), giving 8.82 g of liquid 1-butyl-3-methylimidazolium ethylsulfite. The yield is virtually quantitative. The product is investigated by means of NMR spectroscopy.

¹H NMR (reference: TMS; solvent: CD₃CN), ppm: 0.89 t (CH₃); 1.06 t (CH₃); 1.28 m (CH₂); 1.79 m (CH₂); 3.56 q (CH₂O); 3.87 s (CH₃); 4.18 t (CH₂); 7.47 m (CH); 7.51 m (CH); 9.56 br. s. (CH); ³J_{H,H}=7.1 Hz.

### Example 3

### 1-Butyl-3-methylimidazolium ethylsulfite

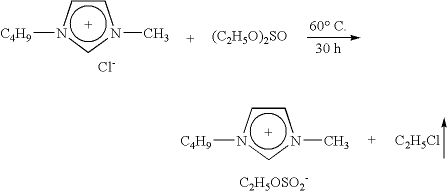

A mixture of 3.14 g (18.0 mmol) of 1-butyl-3-methylimidazolium chloride and 4.97 g (36.0 mmol) of diethyl sulfite is stirred at 60-70° C. (temperature of the oil bath) for 30 hours under an inert-gas atmosphere (nitrogen) in a sealed reaction vessel with pressure valves for 1-1.5 bar above atmospheric pressure. The end of the reaction is determined by NMR measurement. The product is pumped off over the course of 14 hours in vacuo at 13.3 Pa and 60° C. (temperature of the oil bath), giving 4.41 g of liquid 1-butyl-3-methylimidazolium ethylsulfite. The yield is virtually quantitative. The product is investigated by means of NMR spectroscopy.

¹H NMR (reference: TMS; solvent: CD₃CN), ppm: 0.89 t (CH₃); 1.06 t (CH₃); 1.28 m (CH₂); 1.79 m (CH₂); 3.56 q (CH₂O); 3.87 s (CH₃); 4.18 t (CH₂); 7.47 m (CH); 7.51 m (CH); 9.56 br. s. (CH); ³J_{H,H}=7.1 Hz.

### Example 4

### 1-Ethyl-3-methylimidazolium methylsulfite

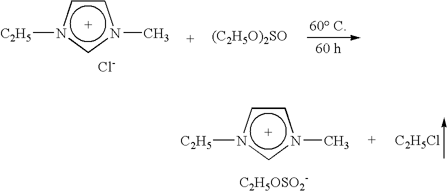

A mixture of 4.90 g (33.4 mmol) of 1-ethyl-3-methylimidazolium chloride and 5.67 g (41.0 mmol) of diethyl sulfite is stirred at 60° C. (temperature of the oil bath) for 60 hours under an inert-gas atmosphere (nitrogen) in a sealed reaction vessel with pressure valves for 1-1.5 bar above atmospheric pressure. The end of the reaction is determined by NMR measurement. The product is pumped off over the course of 2 hours in vacuo at 13.3 Pa and 60° C. (temperature of the oil bath), giving 7.23 g of liquid 1-ethyl-3-methylimidazolium ethylsulfite. The yield is virtually quantitative. The product is investigated by means of NMR spectroscopy.

¹H NMR (reference: TMS; solvent: CD₃CN), ppm: 1.06 t (CH₃); 1.43 t (CH₃); 3.56 q (CH₂O); 3.87 s (CH₃); 4.22 q (CH₂); 7.47 m (CH); 7.53 m (CH); 9.49 br. s. (CH); ³J_{H,H}=7.3 Hz.

### Example 5

### Tetrabutylammonium Methylsulfite

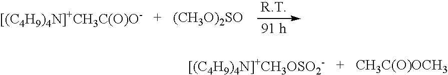

A mixture of 1.84 g (6.1 mmol) of tetrabutylammonium acetate and 0.672 g (6.1 mmol) of dimethyl sulfite is stirred at room temperature for 91 hours under an inert-gas atmosphere (nitrogen) in a sealed reaction vessel. The end of the reaction is determined by NMR measurement. The product is pumped off over the course of 2 hours in vacuo at 13.3 Pa and room temperature, giving 1.98 g of liquid tetrabutylammonium methylsulfite. The yield is virtually quantitative. The product is investigated by means of NMR spectroscopy.

¹H NMR (reference: TMS; solvent: CD₃CN), ppm: 0.95 t (4CH₃); 1.34 t, q (4CH₂); 1.60 m (4CH₂); 3.10 m (4CH₂); 3.15 s (CH₃O); ³J_{H,H}=7.4 Hz.

### Example 6

### Tetrabutylammonium Ethylsulfite

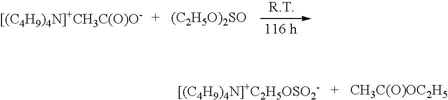

A mixture of 3.69 g (12.2 mmol) of tetrabutylammonium acetate and 0.672 g (12.2 mmol) of dimethyl sulfite is stirred at room temperature for 116 hours under an inert-gas atmosphere (nitrogen) in a sealed reaction vessel. The end of the reaction is determined by NMR measurement. The product is pumped off over the course of 3 hours in vacuo at 13.3 Pa and room temperature, giving 4.19 g of liquid tetrabutylammonium ethylsulfite. The yield is virtually quantitative. The product is investigated by means of NMR spectroscopy.

¹H NMR (reference: TMS; solvent: CD₃CN), ppm: 0.90 t (4CH₃); 1.06 t (CH₃); 1.33 t,q (4CH₂); 1.60 m (4CH₂); 3.13 m (4CH₂); 3.52 q (CH₂O); ³J_{H,H}=7.4 Hz.

### Example 7

### Tetramethylammonium Ethylsulfite

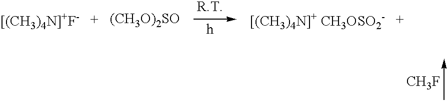

A mixture of 1.34 g (14.4 mmol) of tetramethylammonium fluoride and 3.17 g (28.8 mmol) of dimethyl sulfite is stirred at 70° C. (temperature of the oil bath) for 2 hours under an inert-gas atmosphere (nitrogen) in a sealed reaction vessel with pressure valves for 1-1.5 bar above atmospheric pressure. The end of the reaction is determined by NMR measurement. The excess dimethyl sulfite is pumped off over the course of 2 hours in vacuo at 13.3 Pa and 100° C. (temperature of the oil bath), giving 2.34 g of solid tetrabutylammonium ethylsulfite. The melting point is 155-157° C. The yield is 96.3%. The product is investigated by means of NMR spectroscopy.

¹H NMR (reference: TMS; solvent: D₂O), ppm: 3.05 s (4CH₃); 3.21 s (CH₃O).

### Example 8

### 1,3-Dimethylimidazolium methylsulfite

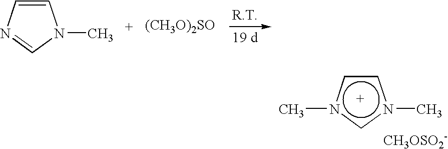

A mixture of 5.89 g (71.7 mmol) of N-methylimidazole and 7.90 g (71.7 mmol) of dimethyl sulfite is stirred at room temperature for 19 days under an inert-gas atmosphere (nitrogen) in a sealed round-bottom flask (50 ml). The end of the reaction is determined by NMR measurement. The product is pumped off over the course of 12 hours in vacuo at 13.3 Pa and room temperature, giving 13.63 g of liquid 1,3-dimethylimidazolium methylsulfite. The yield is virtually quantitative. The product is investigated by means of NMR spectroscopy.

¹H NMR (reference: TMS; solvent: CD₃CN), ppm: 3.18 s (CH₃O); 3.86 s (2CH₃); 7.51 d (2CH); 9.45 br. s. (CH); ⁴J_{H,H}=1.6 Hz.

### Example 9

### Oxidation of 1,3-dimethylimidazolium methylsulfite

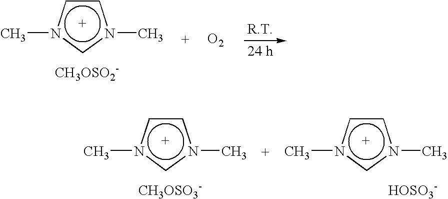

Gaseous oxygen is blown through a solution of 1.33 g of 1,3-dimethylimidazolium methylsulfite in 20 ml of acetonitrile for 24 hours. The solid product is filtered off and pumped off over the course of 1 hour in vacuo at 13.3 Pa and 50° C., giving 0.71 g of solid product, which can be shown to be 1,3-dimethylimidazolium hydrogensulfate by means of NMR and elemental analysis.

¹³C NMR (reference: TMS external; solvent: D₂O), ppm: 36.4 q (2CH₃), 124.0 d (2CH), 137.2 d (CH), ¹J_{C,H}=144 Hz, ¹J_{C,H}=202 Hz, ¹J_{C,H}=223 Hz.

Elemental analysis: found, %: C, 32.08; H, 5.12; N, 14.91; S 17.01. calculated for C₅H₁₀N₂O₄S, %: C, 30.93; H, 5.19; N, 14.42; S 16.51.

Acetonitrile is removed by means of a rotary evaporator, and the product which remains (0.5 g) is investigated by means of NMR spectroscopy. The spectrum is identical to the spectrum of 1,3-dimethylimidazolium methylsulfate.

¹H NMR (reference: TMS; solvent: CD₃CN), ppm: 3.50 s (CH₃O); 3.84 s (2CH₃); 7.39 d (2CH); 8.86 br. s. (CH); ⁴J_{H,H}=1.5 Hz.

1. Process for the preparation of onium alkylsulfites by reaction of an onium halide or carboxylate with a symmetrically substituted dialkyl sulfite or with an asymmetrically substituted dialkyl sulfite at temperatures of 0 to 70° C.

2. Process according to claim 1, characterised in that the reaction is carried out with a symmetrically substituted dialkyl sulfite, where the alkyl group can have 1 to 10 C atoms.

3. Process according to claim 1, characterised in that the reaction is carried out with an asymmetrically substituted dialkyl sulfite, where one alkyl group can have 1 to 10 C atoms and the second alkyl group denotes methyl or ethyl.

4. Process according to claim 1, characterised in that the halide is an ammonium halide, phosphonium halide, thiouronium halide, guanidinium halide or a halide with a heterocyclic cation, where the halides can be selected from the group fluorides, chlorides or bromides.

5. Process according to claim 1, characterised in that the carboxylate is an ammonium acetate, phosphonium acetate, a guanidinium acetate or an acetate with a heterocyclic cation.

6. Process according to claim 1, characterised in that the halide conforms to the formula (1)
[XR₄]⁺Hal⁻ (1)
where
X denotes N, P
Hal denotes F, Cl or Br and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms,
which may be substituted by alkyl groups having 1-6 C atoms,
where one or more R may be partially substituted by Cl, Br and/or CN or partially or fully by F, or F and Cl, or F and Br, or F, Cl and Br, but where all four or three R cannot be fully substituted by halogens,
and where one or two non-adjacent carbon atoms of the R which are not in the α- or ω-position may be replaced by atoms and/or atom groups selected from the group —O—, —C(O)—, —S—, —S(O)— or —SO₂—.

7. Process according to claim 1, characterised in that the halide conforms to the formula (2)
[C(NR¹R²)(NR³R⁴)(NR⁵R⁶)]⁺Hal⁻ (2),
where
Hal denotes F, Cl or Br and
R¹ to R⁶ each, independently of one another, denote hydrogen or CN,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms,
which may be substituted by alkyl groups having 1-6 C atoms,
where one or more of the substituents R¹ to R⁶ may be partially substituted by NO₂, CN, Cl and/or Br, partially or fully by F, or F and Cl, or F and Br or F, Cl and Br, but where all substituents on an N atom cannot be fully substituted by halogens,
where the substituents R¹ to R⁶ may be connected to one another in pairs by a single or double bond and
where, in the substituents R¹ to R⁶, one or two non-adjacent carbon atoms which are not bonded directly to the heteroatom and are not in the ω-position may be replaced by atoms and/or atom groups selected from the group —O—, —C(O)—, —S—, —S(O)— or —SO₂—.

8. Process according to claim 1, characterised in that the halide conforms to the formula (3)
[(R¹R²N)—C(═SR⁷)(NR³R⁴)]⁺Hal⁻ (3),
where
Hal denotes Cl or Br and
R¹ to R⁴ and R⁷ each, independently of one another, denote hydrogen or CN, where hydrogen is excluded for R⁷,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more of the substituents R¹ to R⁴ and R⁷ may be partially or fully substituted by F, Cl and/or Br, in particular —F and/or —Cl, or partially by CN, but where all substituents on an N atom cannot be fully substituted by halogens,
where the substituents R¹ to R⁴ and R⁷ may be connected to one another in pairs by a single or double bond and where, in the substituents R¹ to R⁴ and R⁷, one or two non-adjacent carbon atoms which are not bonded directly to the heteroatom and are not in the w-position may be replaced by atoms and/or atom groups selected from the group —O—, —C(O)—, —S—, —S(O)— or —SO₂—.

9. Process according to claim 1, characterised in that the halide conforms to the formula (4)
[HetN]⁺Hal⁻ (4),
where
Hal denotes Cl or Br and
HetN⁺ denotes a heterocyclic cation selected from the group 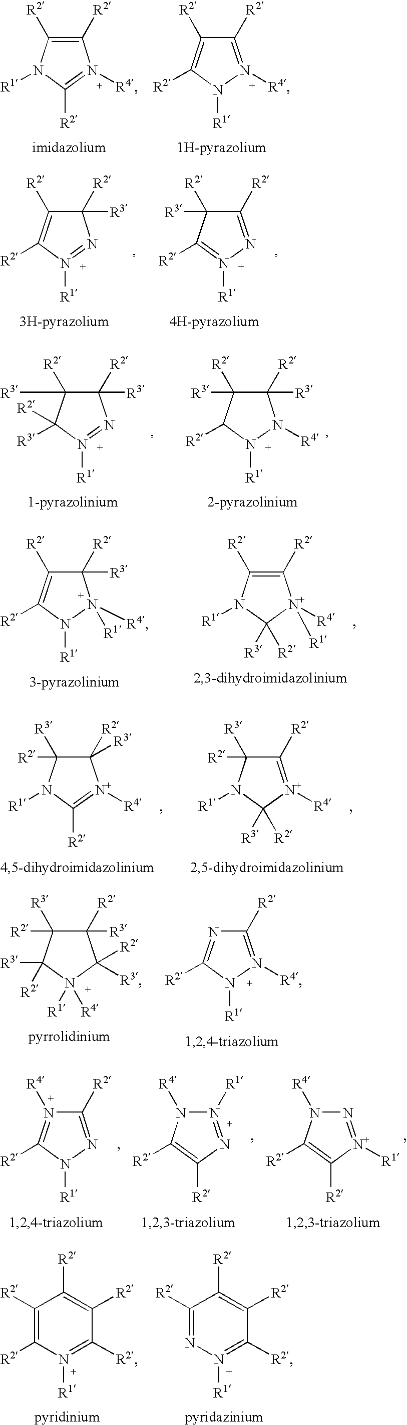 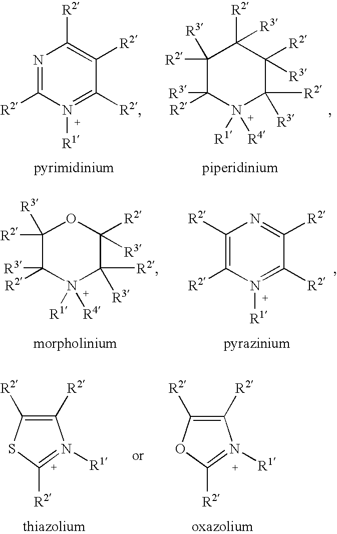
where the substituents
R¹′ to R⁴′ each, independently of one another, denote hydrogen or CN,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
dialkylamino containing alkyl groups having 1-4 C atoms, which, however, is not bonded to the heteroatom of the heterocycle,
saturated, partially or fully unsaturated cycloalkyl having 3-7 C atoms,
which may be substituted by alkyl groups having 1-6 C atoms, or aryl-C₁-C₆-alkyl,
where the substituents R¹′ and R⁴′ may be partially or fully substituted by F, Cl and/or Br, in particular —F and/or —Cl, but where R¹′ and R⁴′ are not simultaneously CN or cannot simultaneously be fully substituted by F or other halogens,
where the substituents R²′ and R³′ may be partially or fully substituted by F, Cl and/or Br, in particular —F and/or —Cl, or partially by NO₂ or CN
and where, in the substituents R¹′ to R⁴′, one or two non-adjacent carbon atoms which are not bonded directly to the heteroatom and are not in the w-position may be replaced by atoms and/or atom groups selected from the group —O—, —C(O)—, —S—, —S(O)— or —SO₂—.

10. Process according to claim 1, characterised in that the reaction of the halide with the dialkyl sulfite is carried out without a solvent.

11. Onium alkylsulfites of the formula (9), obtainable by the process according to claim 1
[Kt]⁺[alkyl-O—SO₂]⁻ (9)
where Kt denotes asymmetrical tetraalkylammonium, symmetrical and asymmetrical phosphonium, guanidinium, thiouronium cations or heterocyclic cations, where N-methylpyridinium sulfite is excluded.

12. Use of compounds according to claim 11 as solvent, solvent additive, heat-transfer medium, reducing agent, antioxidant, phase-transfer catalyst, as extractant, as additive, as surface-active substance, as electrolyte in electrochemical cells, as modifier or as plasticiser.

13. A method comprising employing the onium alkylsulfites of claim 11 as starting material for the synthesis of onium salts with alkylsulfate anions by oxidation.
